(19) United States
(12) Patent Application Publication
(10) Pub. No.: US 2018/0182478 A1
(43) Pub. Date: Jun. 28, 2018

(54) METHOD FOR PATIENT VALIDATION, AND DEVICE FOR USING THE METHOD

(71) Applicant: OPASCA GmbH, Mannheim (DE)

(72) Inventors: Steffen LIEBSCHER, Ludwigshafen (DE); Timo MACHMER, Neuhofen (DE); Alexej SWERDLOW, Ludwigshafen (DE)

(21) Appl. No.: 15/739,542

(22) PCT Filed: Jun. 16, 2016
(86) PCT No.: PCT/DE2016/200279

§ 371 Date: Dec. 22, 2017

(30) Foreign Application Priority Data
Jun. 23, 2015 (DE) 10 2015 211 567.4

(51) Int. Cl. G16H 20/40; G16H 10/60; G06K 9/00

(52) U.S. Cl. CPC G16H 20/40; G06K 9/00288; G16H 10/60

(57) ABSTRACT

A patient validation method before a medical treatment, in particular before performing radiation therapy, is configured such that patient-specific data are recorded and saved in a validation database as reference features, wherein current patient features are ascertained and compared with the reference features before each treatment, and the reference features are updated to adapt to reality as needed, and wherein a release for treatment only happens when the comparison produces a preferably specifiable degree of correspondence.

### BACKGROUND

### Technical Field

The present disclosure relates to a method for patient validation before a medical treatment, in particular before performing radiation therapy. Moreover, the disclosure relates to a device for using the method wherein the term “device” is to be understood in the broadest sense. Finally, at issue is a complete device comprising the individual device components.

### Description of the Related Art

Radiation therapy comprises the treatment of benign and malignant diseases. It is regularly used by specialist physicians for radiology or radiation therapy with the collaboration of medical/technical assistants and specialized medical physicists. Ionizing radiation is directed toward the person, normally based on an irradiation plan given the complex irradiation process. A comprehensive organizational and technical quality assurance aims to ensure that irradiation errors are largely excluded.

Safety in radiation therapy comprises both the safety of the specialist personnel and the safety of patients, including third parties not directly involved in therapy (such as accompanying persons or ambulance personnel). Automated workflows and redundant safety systems should correspond to the safety requirement. A comprehensive solution approach is advantageous in this context, wherein a patient-specific or patient-individualized orientation is sought.

Active safety precautions include patient validation to prevent the irradiation of the wrong patient or switching of patients. The aim is to clearly identify the patient and only provide him to the treatment after successful patient validation, or only to start treatment after the patient's identity is absolutely certain.

In practice, it is already known to record and save patient-specific data. Patient validation is performed only insofar as that the patient is queried, and at most by making visual comparisons, for example using photographic images. Mixups during the hectic hospital and treatment routine are not ruled out.

### BRIEF SUMMARY

The present disclosure specifies a method for patient validation by which the treatment or irradiation of wrong patients, or the mix-up of patients before treatment, is excluded to the greatest possible extent. Moreover, a corresponding device for using the method is specified.

According to various aspects of the disclosure, patient-specific data are recorded and saved in a validation database as reference features for the respective patient. Before each treatment, current patient features are ascertained from the patient, and these are compared with the reference features from the validation database. Release for treatment only happens when the comparison carried out by computer and the algorithms used there produce a correspondence, or at least a high degree of correspondence. The required degree of correspondence can be predetermined by the operator.

The device according to the disclosure is characterized by a computer that accesses a possibly external database having data for patient identification, wherein a second database, a validation database containing the patient-specific data, is accessed by using these data, wherein current, specific patient data are compared with reference features from the validation database, and wherein both the reference features as well as the current patient features are ascertained by sensors, in particular by camera systems.

It is noted at this juncture that this is electronic data processing-assisted patient validation using a peripheral system for recording patient-specific data and a computer that prepares the patient-specific data and supplies them to a validation database.

From there, the patient-specific data can be retrieved for comparative inspection, in particular for a comparison by way of algorithms, namely via a patient identification that can be stored in a separate database such as an external database. The patient identification can, for example, contain the name of the patient and bibliographic data by way of which the patient can be clearly defined.

By way of the patient identification (ID), reference features from the validation database are retrieved and they are compared with current features of the person to be treated, wherein this comparison can also be performed “manually” by an operator, i.e., on a monitor with a corresponding depiction of the reference features.

As already stated, the patient-specific data in the validation database serve for unambiguous patient identification, wherein a corresponding patient ID serves for retrieving the patient-specific data from the validation database. The data can be compiled in this manner.

Advantageously, biometric characteristics serve as patient-specific data, wherein these can be biometric features of the face or body, or individual body parts such as the back or buttocks of a person. The body posture can also be used for identification. The same applies to a fingerprint.

More advantageously, the patient-specific data include photographic images or camera photographs of the patient.

The biometric characteristics are used both to generate the reference features and to determine the current patient features, wherein the determination is camera-based. The data obtained in this manner are processed in a computer and, for example, supplied to the reference database or to the computerized comparison by algorithm.

The appearance of the patient changes especially over the duration of radiation therapy. It is therefore advantageous to adapt the reference features to altered patient features over the phase of treatment, or to update the reference features to match reality. While updating the reference features, it must be ensured that they are actually the features of the same patient.

Most advantageously, the patient validation is automated and is triggered by an event such as the entry into a treatment room or closing a radiation shielding door, etc. Any triggering events can be defined.

In the context of an automated performance of radiation treatment, it is also advantageous when the release for treatment directly or indirectly affects the treatment machine and/or the treatment therapy, i.e., after positive patient validation. An interlock release then occurs so that the radiation process can start when the patient is properly positioned.

More is required than just excluding the treatment of the wrong patient. Rather, a patient with the wrong identity being in the hazard zone of a radiation room, for example, must also be prevented.

For example, it is also conceivable that a door terminal is provided at the entrance area to an irradiation room, from where identity can be ascertained, an analysis can be performed using the patient-specific data/reference features, and finally a release or termination of the operation can occur when it is determined that a patient with the wrong identity is in the hazard zone. In this respect, the preventative measure of a door terminal at the entry area of the treatment/radiation room is highly important.

### BRIEF DESCRIPTION OF THE SEVERAL VIEWS OF THE DRAWINGS

There are now various options for advantageously designing and developing the teaching of the present disclosure. In this regard, reference is made on the one hand to the claims dependent on claim 1, and on the other hand to the following explanation of a preferred exemplary embodiment of the disclosure using the drawings. Generally preferred embodiments and developments of the teaching are also explained in conjunction with the explanation of the preferred exemplary embodiment of the disclosure with reference to the drawings. In the drawings:

FIG. 1 shows the basic process of the method according to the disclosure in a graphic, and

FIG. 2 shows in a flow chart how the method according to the disclosure can proceed in detail.

### DETAILED DESCRIPTION

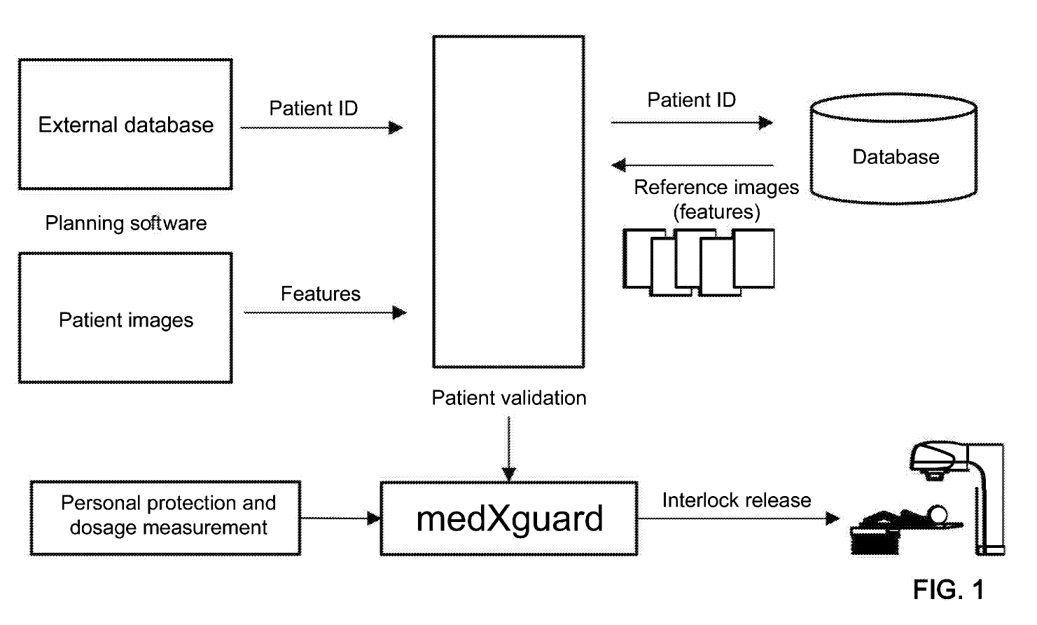

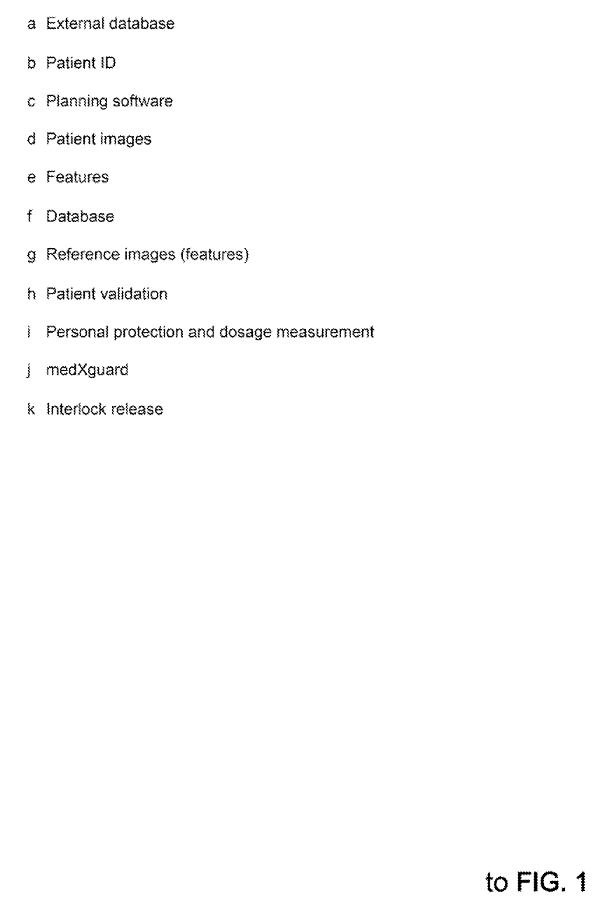

FIG. 1 shows the basic process of a method according to the present disclosure, according to which a patient identification (ID) is saved in a possibly external database. The patient ID can be specifically pulled via the name of the patient, date of birth etc., wherein the patient ID serves to access reference features from a validation database.

The reference features include biometric characteristics as well as one or more photographic images of the patient. With the patient ID, explicit reference features can be pulled from the validation database that are, preferably automatically, compared with current patient features of the patient waiting to be treated, i.e., in the context of electronic data processing-assisted patient validation. It is possible to compare photographic images as well as biometric characteristics in combination, wherein an automated electronic data processing comparison can be carried out on the basis of suitable algorithms. Only after successful patient validation, i.e., after explicit confirmation of the “correct” patient, is there an interlock release with a physician-specified dose rate at the machine.

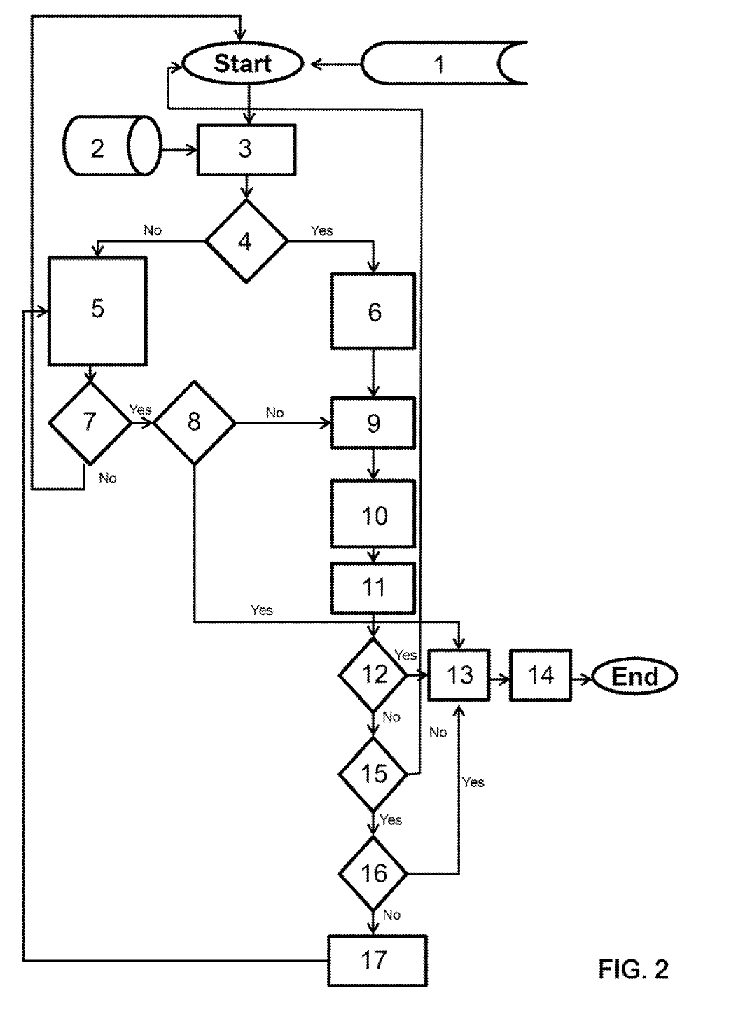

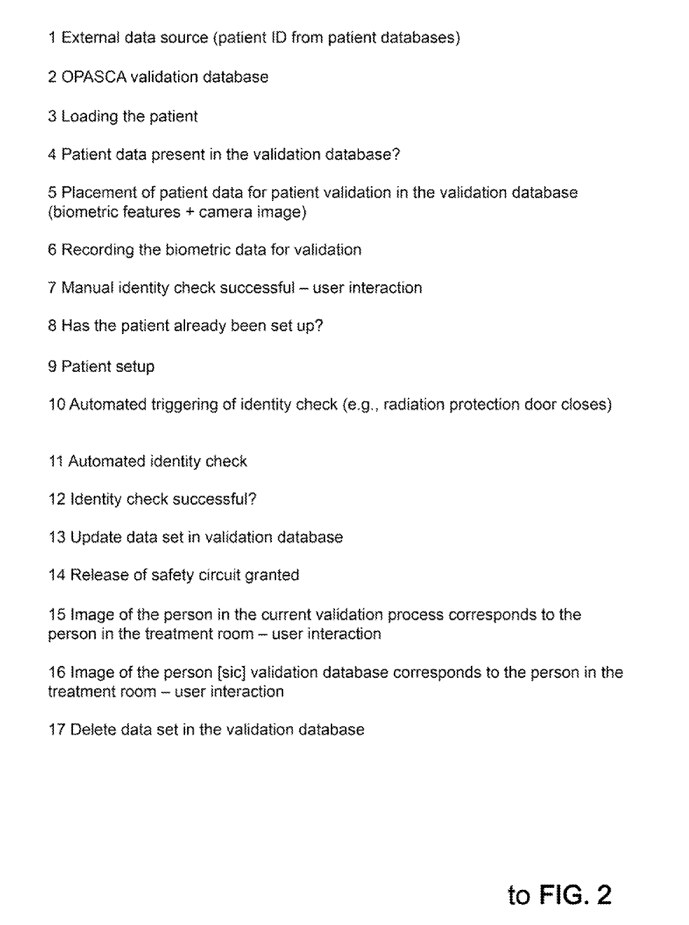

In a flow chart, FIG. 2 shows how the patient validation can occur in the sense of an automated process until a safety circuit is released, after which the radiation procedure can occur. Since the flowchart with the written explanation is self-explanatory, additional details in that regard are superfluous in consideration of the above statements with regard to FIG. 1.

In the context of the method according to the present disclosure, biometric characteristics are ascertained using a suitable peripheral system, for example comprising cameras and camera systems, fingerprint detectors, etc., both to generate patient data for the validation database as well as to ascertain current patient features for comparison with the saved reference features.

It is conceivable to incorporate any desired redundancies and safety criteria in the validation, up to an alternative or conclusive evaluation by an operator, for example at an entry terminal, at the desk of an associated processing room, etc. A significant degree of security can be achieved, and this to the benefit of the patient to be treated, since the mix-up of patients with respect to a medically defined or prescribed irradiation can be effectively almost entirely excluded.

With regard to further advantageous embodiments of the method according to the disclosure, to avoid repetition, reference is made to the general part of the description and accompanying claims.

It is expressly noted in conclusion that the above-described exemplary embodiment of the method according to the disclosure only serves to explain the claimed teaching, which, however, is not restricted to the exemplary embodiment.

1—External data source (patient ID from patient databases)

2—OPASCA validation database

3—Loading the patient

4—Patient data present in the validation database?

5—Placement of patient data for patient validation in the validation database (biometric features + camera image)

6—Recording the biometric data for validation

7—Manual identity check successful—user interaction

8—Has the patient already been set up?

9—Patient setup

10—Automated triggering of identity check (e.g., radiation protection door closes)

11—Automated identity check

12—Identity check successful?

13—Update data set in validation database

14—Release of safety circuit granted

15—Image of the person in the current validation process corresponds to the person in the treatment room—user interaction

16—Image of the person in validation database corresponds to the person in the treatment room user interaction

17—Delete data set in the validation database

The embodiments described above can be combined to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for patient validation before a medical treatment, in particular before performing radiation therapy, comprising:
recording and saving patient-specific data in a validation database as reference features,
ascertaining and comparing current patient features with the reference features before each treatment, and
release the current patient for treatment only when the comparison produces a specifiable degree of correspondence.

2. The method according to claim 1, wherein the patient-specific data of the validation database is addressable or retrievable via a patient identification from an external patient database.

3. The method according to claim 1, wherein biometric characteristics of the current patient serve as patient-specific data.

4. The method according to claim 3, wherein the biometric characteristics are biometric features of the face.

5. The method according to claim 3, wherein the biometric characteristics are biometric features of the body.

6. The method according to claim 1, wherein the patient-specific data comprise a photographic image or a camera picture of the current patient.

7. The method according to claim 3, wherein the biometric characteristics are determined by camera, both to generate the reference features and to ascertain the current patient features, and are processed in a computer.

8. The method according to claim 1, wherein the reference features are adapted to patient features that are altered over the phase of treatments, or the reference features are updated to adapt to reality of the current patient.

9. The method according to claim 1, wherein the patient validation is triggered automatically by an event including entry into a treatment room or closing of a radiation shielding door.

10. The method according to claim 1, wherein the release for treatment directly or indirectly affects the treatment machine and/or the treatment therapy.

11. A device for using a method according to claim 1, comprising a computer that accesses an external database having data for patient identification, wherein a second database, a validation database containing the patient-specific data, is accessed by using these data, wherein current, specific patient data are compared with reference features from the validation database, and wherein both the reference features and the current patient features are ascertained by sensors in camera systems.
